(19) United States
(12) Patent Application Publication
(10) Pub. No.: US 2009/0136594 A1
(43) Pub. Date: May 28, 2009

(54) Functionalization of Micro-And Nano Particles for Selective Attachment to Calcium Biomineral Surfaces

(75) Inventors: Stacey L. McLeroy, Coppell, TX (US); Bruce E. Gnade, Lewisville, TX (US); Jeffrey A. Cadeddu, Dallas, TX (US); Margaret Pearle, Dallas, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/324,718

(22) Filed: Nov. 26, 2008

Related U.S. Application Data
(60) Provisional application No. 60/990,602, filed on Nov. 27, 2007.
(60) Provisional application No. 61/052,452, filed on May 12, 2008.
(60) Provisional application No. 61/102,154, filed on Oct. 2, 2008.

(51) Int. Cl. A61K 33/26; A61M 29/00; A61B 17/28; A61B 19/00; A61B 6/00

(52) U.S. Cl. 424/648; 606/191; 606/205; 128/898; 600/476

(57) ABSTRACT

The present invention includes compositions, methods, devices and kits for magnetizing a biological particle by contacting a biological particle with a ferrous or magnetic particle that is able to specifically bind the biological particle and reacting the biological particle with the ferrous or magnetic particle under physiological conditions, wherein the ferrous or magnetic particle causes the biological particle to become attractable magnetically.

### CROSS-REFERENCE TO RELATED APPLICATIONS

This application claims priority to U.S. Provisional Application Ser. No. 60/990,602, filed Nov. 27, 2007, to U.S. Provisional Application Ser. No. 61/052,452, filed May 12, 2008, and to U.S. Provisional Application Ser. No. 61/102,154, filed Oct. 2, 2008, the contents of each of which is incorporated by reference herein in its entirety.

### TECHNICAL FIELD OF THE INVENTION

The present invention relates in general to the field of biomaterials, and more particularly, to compositions, methods, tools and kits for the functionalization of biomaterials and their selective capture under physiologic conditions.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the functionalization of biomaterials.

Kidney stone disease is a major health problem affecting 5-10% of the U.S. population and accounts for over $2 billion in annual expenses. Compositions and methods are taught herein to assist in the retrieval of biomaterials, e.g., kidney stone fragments, which have been modified to increase magnetic susceptibility. Until the mid 1980s, patients requiring treatment of urinary stones underwent invasive surgical procedures associated with significant surgical morbidity and time lost from work. The introduction of extracorporeal techniques for stone fragmentation and refinements in endoscopic surgery has decreased greatly the morbidity associated with stone surgery without compromising success. However, in up to 30% of cases, residual stone fragments (RSF) remain in the kidney, necessitating secondary surgical procedures and thus increasing morbidity and costs. Furthermore, RSF impact stone recurrence rates, which can reach 80% at 5 years. Therefore, achieving a stone free state remains a surgical and technological challenge.

The kidneys are the organs of urinary filtration and excretion where the urine accumulates into and drains from the renal-collecting system. The urinary filtrate enters the collecting system via the renal papillae where the small collecting tubules coalesce. The urine then travels through a branching system of calyces which coalesce to form a single renal pelvis that then drains into the ureter. As most stones over 5 mm diameter cannot pass through this collecting system, surgical fragmentation and/or extraction is necessary.

In clinical practice, identifying and fragmenting a stone requires that the collecting system be explored in its entirety with flexible endoscopes passed either up the ureter from the bladder or through a small incision in the flank directly into the kidney. Upon fragmentation, innumerable small stone pieces, most less than 2 mm, are generated, making complete clearance of all stone fragments difficult. Clearance is further hindered by scattering of the tiny fragments throughout the collecting system due to fluid irrigation that is used for visualization, by limited ability to access some calyces due to anatomical or technical limitations of the endoscope, and by suboptimal working conditions such as low visibility due to bleeding. Furthermore, retrieving the entire burden of stone fragments is time-consuming, and thus increases operative time and morbidity.

Certain attempts have been made to manipulate cells magnetically, see, e.g., H C Bryant, D A Sergatskov, Debbie Lovato, Natalie L Adolphi, Richard S Larson and Edward R Flynn, Magnetic needles and superparamagnetic cells Phys. Med. Biol. 52 (2007) 4009-4025. Briefly, Superparamagnetic nanoparticles can be attached in great numbers to pathogenic cells using specific antibodies so that the magnetically-labeled cells themselves become superparamagnets. However, the interaction at the cellular level between cell and extracellular matrix, as well as encapsulation within tissue has made this approach to capturing cells challenging to implement.

### SUMMARY OF THE INVENTION

In one embodiment, the present invention includes compositions and method of magnetizing a biological particle by contacting a biological particle with a ferrous or magnetic particle that is able to specifically bind the biological particle; and reacting the biological particle with the ferrous or magnetic particle under physiological conditions, wherein the ferrous or magnetic particle causes the biological particle to become attractable magnetically. In one aspect, the biological particle is a kidney stone or fragment thereof. In another aspect, the ferrous or magnetic particle further includes an agent that specifically binds the surface of the biological target with high affinity such as antibodies, aptamers, peptides, polypetides, proteins, amino acids, polyamino acids, small organic molecules, phosphonic acids, carboxylic acids, long chain phosphonic and long chain carboxylic acids. The biological particle may include at least one of Apatite, Calcium Oxalate Monohydrate, Calcium Oxalate Dihydrate, Brushite Uric Acid, Cysteine and Struvite. In one aspect, the ferrous particle is, e.g., Fe, Fe₂O₃, Fe₃O₄ or FeC. In one specific example, the biological particle comprises a kidney stone or fragment thereof.

In certain aspects, the ferrous or magnetic particle is suspended in a physiological solution, e.g., saline. In one aspect, the biological particles and the ferrous or magnetic particles are located in a anatomical lumen and further comprising the step of removing the biological particle with a device that attracts the ferrous or magnetic particle. In certain examples, the ferrous or magnetic particle may have a size of, e.g., between 5 nm to 1 mm. In certain aspects, the ferrous or magnetic particle may have a size of 5, 10, 50, 100, 200, 250, 350, 400, 500, 600, 750, 800, 900, nanometers or even 1, 5, 10, 25, 50, 200, 250, 350, 400, 500, 600, 750, 800, 900 micrometers or even 1 mm. In one aspect, the ferrous or magnetic particle further comprises an agent that specifically binds the surface of the biological target selected from proteins that interact with calcium-based biominerals, such as carboxylic acid-rich proteins, osteopontin, Tamm-Horsfall protein, and prothrombin fragment 1; small carboxyl-containing molecules such as citrate are known to have an inhibitory effect on the growth of Calcium Oxalate crystals in healthy urine; and calcium-binding protein motifs that includes the EF-hand, which have carboxylic-rich residues (Glu, Asp) and Glycine for flexibility; and short peptides that are rich in polar or charged residues.

In another embodiment, the present invention includes a medical device having, e.g., an expansible member having a proximal end, a delivery state, and a deployed state; wherein the deployed state comprises an at least partially magnetic portion for deployment within an anatomical lumen for the capture of a magnetic target material, and wherein the deployed state is configured to retrieve the material from within the anatomical lumen. In one aspect, the device may also have an elongated flexible tube including a distal end and a proximal end, the tube defining a channel extending from the proximal end of the tube to an aperture at the distal end and wherein the deployed portion of the member is housed within the channel prior to deployment within the anatomical lumen. In one aspect, the delivery state is a compressed state. In another aspect, the device extends proximally out of the channel and is configured to control axial movement of the expansible member relative to the tube. In one example, the portion of the medical device that attracts the coated kidney stone can be an attachment to medical devices that are presently used to view and/or collect kidney stones. The attachment can be part of a kit that includes the kidney stone-binding particles and may optionally include irrigation solutions, written materials that describe the methods for use of the materials and the attachment of the functionalized kidney-tone attracting device.

In one aspect, the deployed state is further defined as expansible, wherein the expansible state that has a proximal end and a distal end, and markers are positioned proximate at least one of the distal and proximal ends of the expansible member. In another aspect, the expansible member has a tapered proximal end to facilitate releasable engagement with a distal end of the instrument. In yet another aspect, the expansible member includes a protrusion at the proximal end to facilitate engagement between the instrument and the expansible member. The expansible member may include a material that exhibits an expansion/compression size ratio of approximately 10:1. Examples of expansible member may be made from a biocompatible polymer, plastic, nylon, polyester, or metal. In one example, the expansible member comprises a cavity. The expansible member may also define one or more holes formed therein for passing irrigation therethrough in the deployed state. In one aspect, the device further includes grasping forceps, a collapsible basket, a hook, a net, a lasso, or a sponge. In yet another embodiment, the expansible member expands to fill a cross-sectional area of an anatomical lumen in the expanded, deployed state.

The present invention also includes compositions and methods for immobilizing a magnetic biological material in a body by inserting a magnetic expansible member into an anatomical lumen of the body, the expansible member having a delivery state, an expanded state, and a proximal end detachably engaged with a distal end of an instrument; positioning the instrument to deploy the expansible member such that the expansible member transforms from the delivery state to the expanded state at a treatment site within the anatomical lumen; and capturing biological particles within the lumen that have been modified in situ to be attracted magnetically. In one aspect, the method also includes inserting an expansible member includes providing an elongated flexible tube including a distal end and a proximal end, the tube defining a channel extending from the proximal end of the tube to an aperture at the distal end, and wherein the expansible member is housed within the channel prior to deployment at a treatment site. In one aspect, the step of positioning the instrument is used to deploy the expansible member and includes moving the instrument relative to the tube to control axial movement of the expansible member beyond the channel. In one aspect, the expansible member includes a material that expands to the expanded state when unrestrained. In one aspect, the expansible member is deployed distally beyond the material to be immobilized such that the expansible member at least partially occludes the anatomical lumen. In another aspect, the method further includes the step of performing a lithotripsy procedure on the biomaterial. In one aspect, the method may also include the step of irrigating the anatomical lumen with a composition comprising a ferrous or magnetic particle that is able to specifically bind the biological material. In one aspect, the method also includes the step of retrieving the immobilized biological material by proximally pulling the expansible member through the anatomical lumen. In one aspect, the anatomical lumen includes an interior surface and the expansible member expands to contact the interior surface of the anatomical lumen. In another aspect, the expansible member has a proximal end and a distal end, and markers are positioned proximate the distal and proximal ends of the expansible member. In yet another aspect, the step of positioning further includes visualizing the position of the markers through a medical imaging device. In one aspect, the step of retrieving the immobilized material also includes engaging fragmented immobilized material with the device.

In yet another embodiment, the present invention includes devices and methods for stabilizing a target biomaterial in a patient's body by contacting a first material comprising a magnetic portion with a target biomaterial to cause the biomaterial to become magnetic in a body lumen under physiologic conditions; inserting a medical device comprising a magnetically attracting end, wherein the magnetized biomaterial is attracted to the medical device; and removing the biomaterial from the patient's body. In one aspect, the target biomaterial is broken into at least two fragments by technique selected from the group consisting of extra-corporeal shock wave lithotripsy, intra-corporeal shock wave lithotripsy, or Holmium laser fragmentation.

In another embodiment, the present invention includes a kit that includes one or more containers comprising a ferrous or magnetic particle that is able to specifically bind to a biological particle, and one or more devices comprising an end that is magnetic and able to attract the ferrous or magnetic particle in a body lumen.

In another embodiment, the present invention includes a method of magnetizing and moving a biological particle that includes contacting a biological particle with a ferrous particle that is able to specifically bind the biological particle; reacting, adsorbing, or adhering the biological particle with the ferrous particle under physiological conditions, wherein the ferrous or magnetic particle causes the biological particle to become attractable magnetically; and directing a magnet, e.g., an external or internal magnet or magnetic field, to the location of the ferrous particle to move the particle using the magnetic field generated by the magnet. In one aspect, the biological particle is a kidney stone or fragment thereof. In another aspect, the ferrous particle includes an agent that specifically binds the surface of the biological target with high affinity such as antibodies, aptamers, peptides, polypeptides, proteins, protein fragments, amino acids, polyamino acids, phosphonic acids, carboxylic acids, long chain phosphonic and long chain carboxylic acids. The biological particle may be, for example, at least one of Apatite, Calcium Oxalate Dihydrate, Calcium Oxalate Monohydrate, Struvite, Cysteine, Brushite and Uric Acid. In one aspect, the ferrous particle includes Fe₂O₃ or Fe₃O₄. In yet another aspect, the biological particles and the ferrous or magnetic particles are located in a anatomical lumen and further comprising the step of removing the biological particle with a device that attracts the ferrous or magnetic particle.

Yet another embodiment, the present invention includes a method of identifying a biological particle comprising: contacting a biological particle with a ferrous particle that is able to specifically bind the biological particle and a fluorescent dye; reacting, adsorbing, or adhering the biological particle with the ferrous particle under physiological conditions, wherein the ferrous or magnetic particle causes the biological particle to become attractable magnetically and fluorescent; directing a magnet to the location of the ferrous particle to move the particle using the magnetic field generated by the magnet; and pointing a light that excites the fluorescent dye to provide visualization of the particles.

Another embodiment of the present invention includes a method of identifying a biological particle by contacting a biological particle with a ferrous particle that is able to specifically bind the biological particle and a dye; reacting, adsorbing, or adhering the biological particle with the ferrous particle under physiological conditions, wherein the ferrous or magnetic particle causes the biological particle to become attractable magnetically; directing a magnet to the location of the ferrous particle to move the particle using the magnetic field generated by the magnet; and pointing a light that excites the dye to provide visualization of the particles. In one aspect, the dye comprises a fluorescent dye and/or a visible dye. In another aspect, the dye comprises a fluorescent dye selected from the group of Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide (Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide (PI) and derivatives thereof and Cy-dyes derivatives. Yet another aspect includes a dye selected from a group consisting of blue fluorescent protein (BFP), green fluorescent protein (GFP), photo activatable-GFP(PA-GFP), yellow shifted green fluorescent protein (Yellow GFP), yellow fluorescent protein (YFP), enhanced yellow fluorescent protein (EYFP), cyan fluorescent protein (CFP), enhanced cyan fluorescent protein (ECFP), monomeric red fluorescent protein (mRFP1), kindling fluorescent protein (KFP1), aequorin, autofluorescent proteins (AFPs), JRed, TurboGFP, PhiYFP and PhiYFP-m, tHc-Red (HcRed-Tandem), PS-CFP2 and KFP-Red. The method may also include the step of vacuuming dyed kidney stone dust and/or illuminating the surgical field with polarized light to maximize the visualization of dyed kidney stone dust.

### BRIEF DESCRIPTION OF THE DRAWINGS

For a more complete understanding of the features and advantages of the present invention, reference is now made to the detailed description of the invention along with the accompanying figures and in which:

FIG. 1. FTIR Spectrum of COM Crystals. GATR-FTIR confirms the presence of Calcium Oxalate Monohydrate. Sparse surface area coverage of grown crystals account for differences in signal strength.

FIG. 2. Crystals exhibit typical COM morphology. The most prevalent crystal forms show a coffin-shaped {100} face, and a less common form twinned along the {010} face³.

FIG. 3. Glutamic Acid

FIG. 4. Aspartic Acid

FIG. 5. Calmodulin with four EF-Hand-motifs. Ca²⁺: blue, alpha-helices: orange, beta-sheets: green

FIG. 6. Citrate contains three carboxyl groups.

FIG. 7. Twinned COM crystal dangling from TEM grid

FIG. 8. COM Crystal with citrate coated iron oxide nanoparticles

FIG. 9. HRTEM image of iron oxide nanoparticle adhering to COM crystal

FIG. 10. COM Crystal with —COOH functionalized nanoparticles

FIG. 11. TEM image of COM crystal at edge of copper TEM grid

FIG. 12. FTIR confirmation of peptide functionalization of iron oxide particles

FIG. 13. (a) The carboxyl functional group on the commercially available iron oxide has a slight affinity for COM crystals. (b) The PAA-coupled particles show a much higher attachment rate.

FIG. 14. AGM is used to quantify Super-Paramagnetic Iron Oxide (SPIO) attachment to human kidney stone samples. (a) Diamagnetic behavior of kidney stone powder (60% COM, 35% brushite, 5% apatite); Mₛₐₜ=−0.86 emu/gram (b) Superparamagnetic behavior of iron oxide particles; Mₛₐₜ=29.3 emu/gram (c) Superparamagnetic behavior of SPIO-exposed kidney stones; Mₛₐₜ=0.44 emu/gram

FIG. 15. UV-Vis Spectra of poly-amino acid etching.

FIG. 16. Artificially-grown COM crystals etched in poly-amino acids solutions for 30 minutes. Relative etch rates agree with UV-Vis trend for human COM kidney stones.

FIG. 17. UV-Vis Spectra of Etching vs. Concentration.

FIGS. 18A and 18B show the effects a magnet on kidney stones in solution coated with the compositions of the present invention.

FIGS. 19A and 19B show that a magnet is able to attract and lift kidney stones in solution coated with the compositions of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Kidney stone disease is a major health problem affecting 5-10% of the U.S. population and accounts for over $2 billion in annual expenses. Compositions and methods are taught herein to assist in the retrieval of biomaterials, e.g., kidney stone fragments, which have been modified to make them ferromagnetic or paramagnetic. The compositions, methods, devices and kits taught herein can selectively attach magnetic or ferrous particles to kidney stone fragments using techniques developed for self-assembled monolayer (SAM) formation and the same may be removed by an intraluminal device that attracts the modified biomaterial.

Until the mid 1980s, patients requiring treatment of urinary stones underwent invasive surgical procedures associated with significant surgical morbidity and time lost from work. The introduction of extracorporeal techniques for stone fragmentation and refinements in endoscopic surgery has greatly decreased the morbidity associated with stone surgery without compromising success. However, in up to 30% of cases, residual stone fragments (RSF) remain in the kidney, necessitating secondary surgical procedures and thus increasing morbidity and costs. Furthermore, RSF impact stone recurrence rates, which can reach 80% at 5 years. Therefore, achieving a stone free state remains a surgical and technological challenge.

The kidneys are the organs of urinary filtration and excretion where the urine accumulates into and drains from the renal-collecting system. The urinary filtrate enters the collecting system via the renal papillae where the small collecting tubules coalesce. The urine then travels through a branching system of calyces which coalesce to form a single renal pelvis that then drains into the ureter. As most stones over 5 mm diameter cannot pass through this collecting system, surgical fragmentation and/or extraction is necessary. In clinical practice, identifying and fragmenting a stone requires that the collecting system be explored in its entirety with flexible endoscopes passed either up the ureter from the bladder or through a small incision in the flank directly into the kidney. Upon fragmentation, innumerable small stone pieces, most less than 2 mm, are generated, making complete clearance of all stone fragments difficult. Clearance is further hindered by scattering of the tiny fragments throughout the collecting system due to fluid irrigation that is used for visualization, by limited ability to access some calyces due to anatomical or technical limitations of the endoscope, and by suboptimal working conditions such as low visibility due to bleeding. Furthermore, retrieving the entire burden of stone fragments is time-consuming, and thus increases operative time and morbidity. Thus, an ability to attract these fragments into easily accessible areas of the collecting systems would greatly facilitate endoscopic stone surgery as well as diminish costs by eliminating secondary procedures and reducing stone recurrences.

Micro and nanoparticle technology was used to develop the compositions, methods, devices and kits to facilitate endoscopic stone fragment retrieval. The chemistry and physics of stone formation has been well studied. One of the key questions to be answered in this study is the surface chemistry of the stones in situ. Specifically, novel magnetic nano and microparticles were made that selectively adhere to, e.g., the calcium oxalate crystalline structure of a stone and its fragments. By introducing of a magnetized wire or working instrument (e.g. stone basket), dispersed stone fragments will be attracted and gathered in a favorable location in the renal pelvis for extraction. It is anticipated that this revolutionary technology will significantly improve patient safety and reduce operative time and cost by “bringing the stone to the surgeon, rather than the surgeon to the stone.”

The present invention may use any of a number of magnetic materials to modify the biomaterial surfaces. For example, a wide variety of permanent magnetic materials may be used with the present invention such as rare earth magnets, ceramic magnets, alnico magnets, which may be rigid, semi-rigid and flexible magnets. For use with a medical device, flexible magnets are made by impregnating a flexible material such as neoprene rubber, vinyl, nitrile, nylon or a plastic with a material such as iron flakes having magnetic characteristics and will find use with the present invention. Conversely, the medical device may be rendered magnetic and the material embedded or placed into a base for attachment of the biomaterial may be magnetic. The skilled artisan will recognize that any of a number of magnetic particles may be used, for example, magnetic particles, including Cobalt, Nickel, FePt, SmCo, CoFe, Fe, CoNi, FeCoPt, as well as ceramic materials. Particles may be made from magnetic particles embedded in a non-magnetic or diamagnetic matrix, e.g. silica or polystyrene. Particles of any shape or form, including spherical, nano-crystalline, polycrystalline, cubic, rod-shaped, wire-shaped, or irregular particles may be used. Generally, paramagnetic materials will be used. As used herein, a paramagnetic material refers to a material that only has a magnetic moment in the presence of an magnetic field. However, other magnetic materials may also be used with the present invention.

The medical devices of the present invention may be made from a wide variety of materials that are, e.g., metallic or non-metallic or magnetic or non-magnetic or elastomeric or non-elastomeric or malleable or non-malleable or the one or more second restraints are metallic or non-metallic or magnetic or non-magnetic or elastomeric or non-elastomeric or malleable or non-malleable so long as the tip or distal end includes a magnetic portion. In certain instances the shaft that includes the medical device may be magnetic. For certain embodiments of medical devices (surgical instruments), it is also possible to use magnetic shape-memory alloys and electromagnets.

The present invention may be made such that the base is metallic or non-metallic or magnetic or non-magnetic or elastomeric or non-elastomeric or malleable or non-malleable. Examples of materials include metals, plastics, polymers, wood, alloys, composites and the like. The metals may be made from one or more metals, such as steel, stainless steel, aluminum, titanium, nickel, magnesium, or any other structural metal. Non-limiting examples of plastics or polymers may include: nylon, polyethylene (PE), polypropylene (PP), polyester (PE), polytetrafluoroethylene (PTFE), acrylonitrile butadiene styrene (ABS), polyvinylchloride (PVC), or polycarbonate, for example, GE's Lexan® polycarbonate, and combinations thereof, among other plastics. The tool restraint taught herein may be molded, sintered, machined and/or combinations thereof to form the required pieces to assemble the tool restraint components.

The present invention includes attaching magnetic particles to kidney stone fragments for stone retrieval. While commercial products include functionalized magnetic particles, these materials have not been optimized for physiologic use and medical devices (surgical instruments) have not been made or optimized to collect the magnetized stones. Furthermore, it is possible to semi-permanently or permanently trap the paramagnetic particles onto the target biomaterial by using, e.g., a biological coating (e.g. biotin-streptavidin), polymeric, light or UV-curable polymer to permanently bind the kidney stones. It is also possible to semi-permanently or permanently trap the paramagnetic particles onto the target biomaterial by using, e.g., a biological coating (e.g. biotin-streptavidin), polymeric, light or UV-curable polymer to permanently bind the kidney stones once they make contact with the instrument.

Magnetic particle size. Commercially available iron oxide particles for magnetic cell separations may be used that are as small as 50 nm and as large as 5 microns, with larger particles having more magnetic attraction and thus faster separations.

### EXAMPLE 1

### Development of Magnetic Particles

Magnetic nanoparticles were developed that bond preferentially to the calcium oxalate crystalline structure of most kidney stones. The use of magnetic nanoparticles in medicine was recently reviewed. Typical applications include imaging and tumor treatment. In our case, once magnetic particles are attached, the stone fragment can be attracted to a magnetic tool (wire or stone basket) and moved as needed. Particles of Fe₂O₃ or Fe₃O₄ can be used with no known toxic effects on human tissues. Nevertheless, because of some uncertainty of the toxic effects of these particles, an attempt to prevent passage into general circulation is advisable. Therefore, the nanoparticles will be designed to exceed the diameter of capillary vessels (7 to 8 μm, which is the diameter of a red blood cell) and possibly collecting ducts (40 to 200 μm). There are several processes that have been developed to produce magnetic nanoparticles, but in general these particles are in the size of tens of nanometers.

A physical model was developed to estimate the number of magnetic particles that must be attached as a function of fragment size to generate enough force to attract the fragment over a given distance in a fluid medium. This model provides insight into optimizing both the magnetic particle size as well as the attachment chemistry.

Next, a process was developed to attach magnetic particles to kidney stone fragments. The goal is to functionalize magnetic particles with a molecule that will selectively attach to the kidney stone fragment. There is considerable work in the literature on the self-assembly of dense molecular films on oxide surfaces. Two common functional groups that selectively attach to oxide surfaces are phosphonic acids and carboxylic acids. SAMs can be built on representative surfaces of kidney stone fragments using long chain phosphonic and carboxylic acids. Both human stone fragments extracted at surgery and commercially available CaOx stone phantoms were used to develop the attachment process.

The first part of the project is to determine the surface chemistry of typical kidney stones. X-ray photoelectron spectroscopy (XPS), grazing angle total reflection FTIR (GATR-FTIR), and biochemical assays are used to determine the surface chemistry. Next, whether the surface of the fragment is an oxide, a hydroxide, or is covered with an organic residue is determined. One challenge of preparing surfaces is that they are representative of kidney stones that are in vivo in order to determine the surface chemistry. The next step is to determine which chemistry will selectively build a SAM on the stone surface in a solution that is indicative of the solution in a kidney. The surface coverage of the SAM is measured using XPS, GATR-FTIR and fluorescence measurements. Finally, the kinetics for film formation is determined by measuring the surface coverage as a function of concentration and time in solution, because there will be limited time for film formation during the kidney stone removal procedure.

The next step is to determine the optimal functionalization of the surface of the magnetic particles. Magnetic particles may be purchased or made that can be used to determine the optimal particle size, material and shape. Phosphonic and carboxylic acid functional groups can attach molecules to the magnetic nanoparticles and the target biomaterial, since they will have an oxide surface. Binding of the target under physiologic conditions is required for optimal patient treatment in body lumens. In one example, bi-functional molecules are used to selectively attach the magnetic particles or the ferrous particle to the kidney stone fragment. Examples of groups that may be used to bind the kidney stone target include, e.g., an antibody, a peptide, small oligomers (amino acids, lipids, carbohydrates, aptamers, small molecules or nucleic acids) that specifically bind the target. The functionalized magnetic particles are introduced into the lumen and the kidney stone fragments are removed by a magnetically attracting medical device or tool that is inserted into the lumen and that attracts magnetically the modified biomaterial that has been rendered magnetic. As the skilled artisan will readily recognize, the binding pair will generally include a material that is ferrous and a material that is magnetic. Either the biomaterial or the medical device can be ferrous or magnetic and vice versa. The medical device may also include structures that, in addition to the magnetic attraction, may be used for visualizing the target, capture the kidney stone fragments, provide light to the area, permit fluid flow to deliver the functionalized magnetir or ferrous particles or may be used for irrigation.

The number of magnetic particles attached to the stone fragments as a function of surface area using SEM and EDAX (energy dispersive X-ray analysis) was determined. Once the magnetic particles are attached to the kidney stone fragments, we will determine the efficiency of attracting the stone fragments to a magnetic wire as a function of fragment size, magnetic particle size, magnetic particle number density, distance between fragment and magnetic wire, viscosity of medium, etc. Certain magnetizing particles may obtained from commercial sources, e.g., Sigma, Bioclone, ESPI, Chemicell, Bangs Labs and Invitrogen.

TABLE 1
Magnetic Modification of Biomaterials

| | | | Surface Functional | | | Attract |
|---|---|---|---|---|---|---|
| Designation | Shape | Composition | Group | Size | | Stones? |
| Fe3O4 | nanocrystalline | 100% Fe3O4 | none | 25 | nm | no |
| nanopowder | | | | | | |
| Fe2O3 | nanocrystalline | 100% Fe2O3 | none | 25 | nm | no |
| nanopowder | | | | | | |
| Fe2O3 | nanocrystalline | 100% Fe2O3 | none | 1-5 | um | no |
| micropowder | | | | | | |
| Fe3O4 | nanocrystalline | 100% Fe3O4 | none | 44 | um and | no |
| micropowder | | | | | smaller | |
| A | spherical | Silica-coated iron oxide | Long arm —COOH | 1 | um | yes |
| | | (60%) | | | | |
| B | spherical | 60% iron oxide | —COOH | 1 | um | yes |
| | | polymerized into styrene | | | | |
| | | matrix | | | | |
| C | spherical | 60% iron oxide | —COOH | 1 | um | some |
| | | polymerized into styrene | | | | |
| | | matrix | | | | |
| D | spherical | maghemite | anionic surface | 200 | nm | some |
| E | spherical | magnetite | Poly-DL-aspartic acid, | 200 | nm | few |
| | | | sodium salt | | | |
| F | spherical | magnetite | starch with phosphate | 200 | nm | yes |
| | | | groups | | | |
| G | irregular | 100% iron oxide | — | 10 | um | no |
| H | spherical | iron oxide in styrene | —COOH | 2.7 | um | yes |
| | | matrix core, glycidyl | | | | |
| | | ether, shell | | | | |
| I | spherical | Polymer-based | —COOH | 2.7 | um | some |
| J | spherical | magnetite | cationic surface | 200 | nm | yes |
| K | irregular | 90% iron oxide | —COOH | 1.5 | um | some |
| L | irregular | 90% iron oxide | —COOH | 3-12 | um | few |
| M | spherical | 40% iron oxide in | —COOH | 1.6 | um | few |
| | | styrene matrix core, | | | | |
| | | polymer shell | | | | |
| N | spherical | Silica-coated iron oxide | Short-Arm —COOH | 1 | um | few |
| | | (60%) | | | | |
| P | spherical | magnetite | Phosphate-starch | 200 | nm | some |
| Q | spherical | magnetite | Oleic acid | 200 | nm | no |
| R | spherical | magnetite | Carboxymethyldextran | 200 | nm | Some |
| T | spherical | 60% iron oxide | Poly Aspartic Acid | 1 | um | yes |
| | | polymerized into styrene | | | | |
| | | matrix | | | | |

TABLE 2
Magnetic Attraction Measurements.

| | Stone | | | Sample | | Sample | Mass of | Sample | Particle |
|---|---|---|---|---|---|---|---|---|---|
| Stone | Fragment | Magnetic | Attract | Mass | Sample Mₛₐₜ | Coercivity | Particles | Mₛₐₜ | Msat |
| Composition | Size | Particles | Stones? | (mg) | (emu/gram) | (Oe/mg) | attached | (memu) | (emu/gram) |
| 65% COM | 0.25-0.5 mm | None | N/A | 0.182 | −0.861 | −4041 | | −0.1569 | |
| 65% COM | 0.25-0.5 mm | M | few | 1.034 | −0.067 | −111 | −1.7E−06 | −0.0688 | 40 |
| 65% COM | 0.25-0.5 mm | K | some | 0.767 | −0.028 | 98 | −4.9E−07 | −0.0213 | 43.4 |
| 65% COM | 0.25-0.5 mm | G | no | 2.543 | −0.009 | −232 | −9.6E−07 | −0.023 | 23.8 |
| 65% COM | 0.25-0.5 mm | D | some | 0.628 | 0.033 | 16 | 8.8E−07 | 0.0207 | 23.4 |
| 65% COM | 0.25-0.5 mm | L | few | 0.661 | 0.010 | 93 | 1.4E−07 | 0.0069 | 50.2 |
| 65% COM | 0.25-0.5 mm | E | few | 0.352 | 0.055 | 72 | 1.4E−06 | 0.0193 | 14.0 |
| 65% COM | 0.25-0.5 mm | P | some | 0.987 | 0.027 | 57 | 4.7E−07 | 0.0266 | 57 |
| 65% COM | 0.25-0.5 mm | Q | no | 0.419 | 0.063 | 95 | 4.7E−07 | 0.0266 | 57 |
| 65% COM | 0.25-0.5 mm | R | some | 0.182 | 0.166 | 1903 | 5.3E−07 | 0.0303 | 57 |
| 65% COM | 0.25-0.5 mm | I | some | 0.670 | 0.023 | 72 | 1.5E−07 | 0.0151 | 99.7 |
| 65% COM | 0.25-0.5 mm | J | yes | 0.250 | 0.196 | 246 | 5.5E−06 | 0.049 | 9.0 |
| 65% COM | 0.25-0.5 mm | C | some | 0.683 | 0.171 | 18 | 3.0E−05 | 0.117 | 4.0 |
| 65% COM | 0.25-0.5 mm | A | yes | 0.741 | 0.133 | 16 | 3.7E−05 | 0.0989 | 2.7 |
| 65% COM | 0.25-0.5 mm | S | some | 0.676 | 0.205 | 62 | 3.2E−06 | 0.1386 | 43.4 |
| 65% COM | 0.25-0.5 mm | B | yes | 0.423 | 0.436 | 46 | 2.9E−07 | 0.1845 | 639.7 |
| 65% COM | 0.25-0.5 mm | H | yes | 0.750 | 0.281 | 5 | 1.1E−04 | 0.2104 | 1.9 |
| 95% COM | 0.1-0.25 mm | B | Some | — | — | — | — | — | — |
| 95% COM | 0.1-0.25 mm | T | Yes | — | — | — | — | — | — |
| 60% COM | 0.1-0.25 mm | B | Some | — | — | — | — | — | — |
| 60% COM | 0.1-0.25 mm | T | Yes | — | — | — | — | — | — |
| 100% Uric Acid | 0.1-0.25 mm | T | No | — | — | — | — | — | — |
| 100% Uric Acid | 0.1-0.25 mm | B | No | — | — | — | — | — | — |

### EXAMPLE 2

### Rendering Biomaterials Magnetic

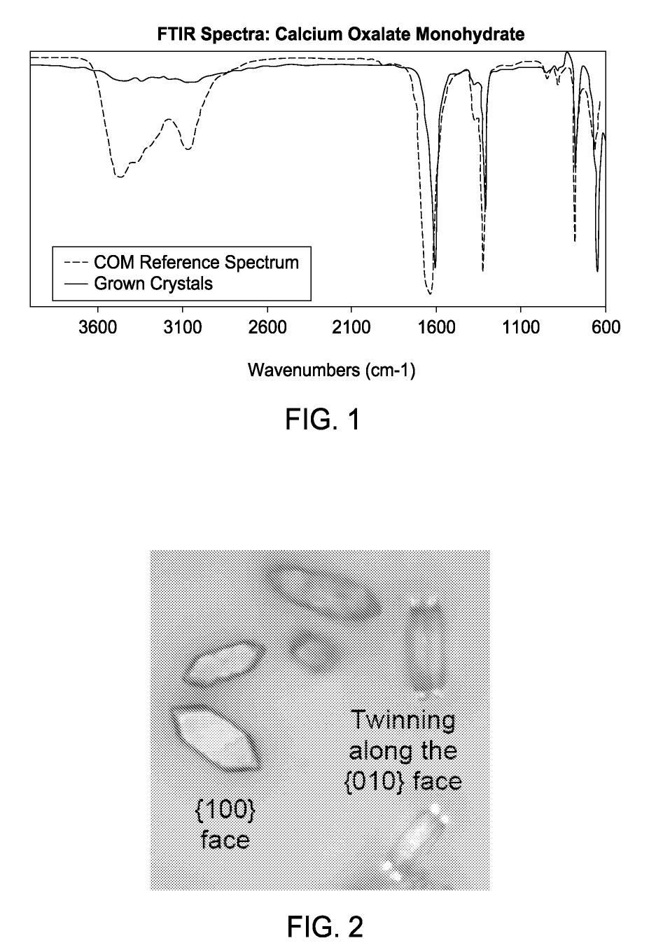

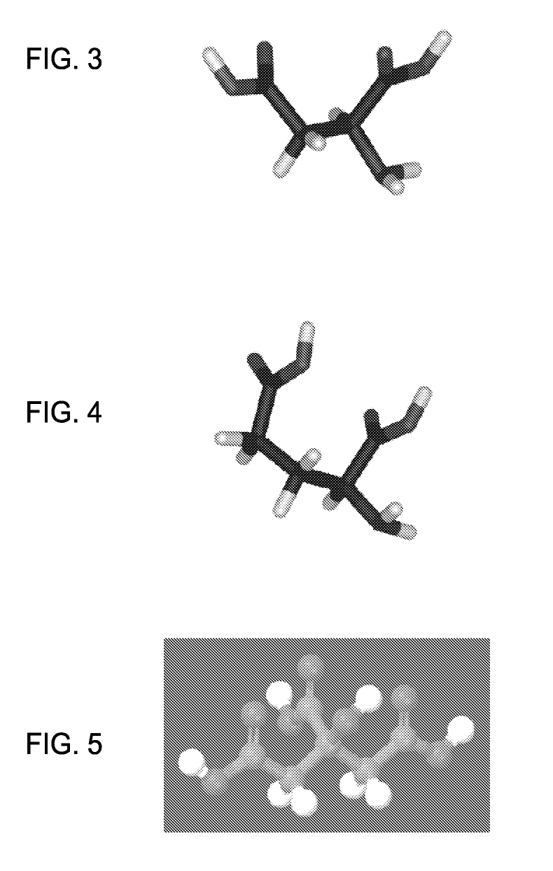

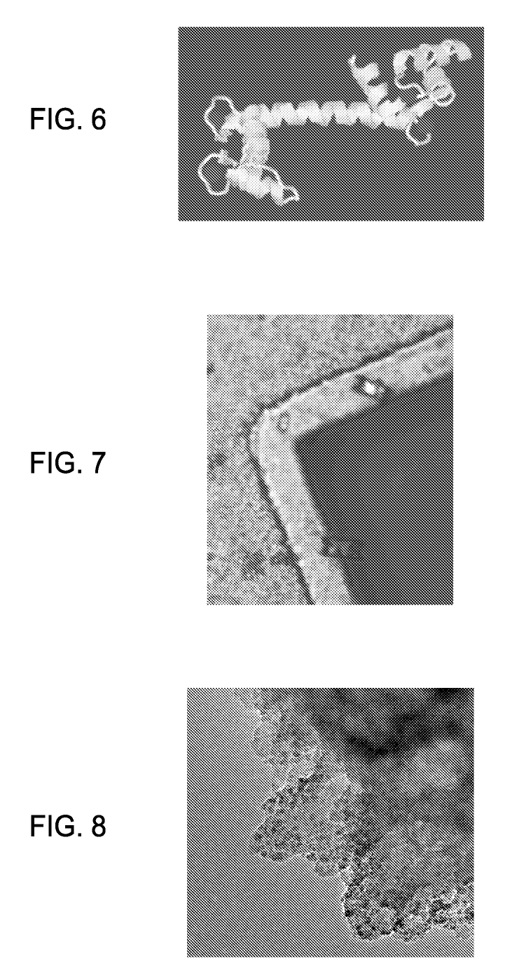

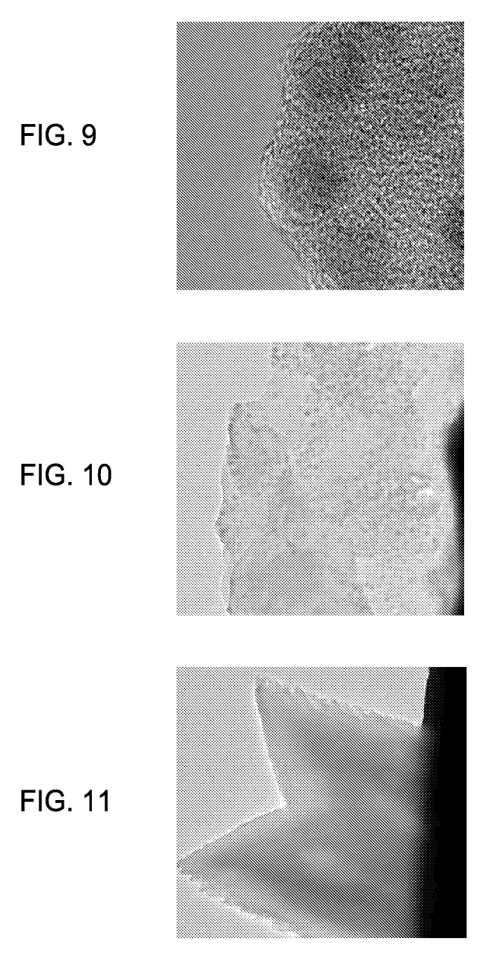

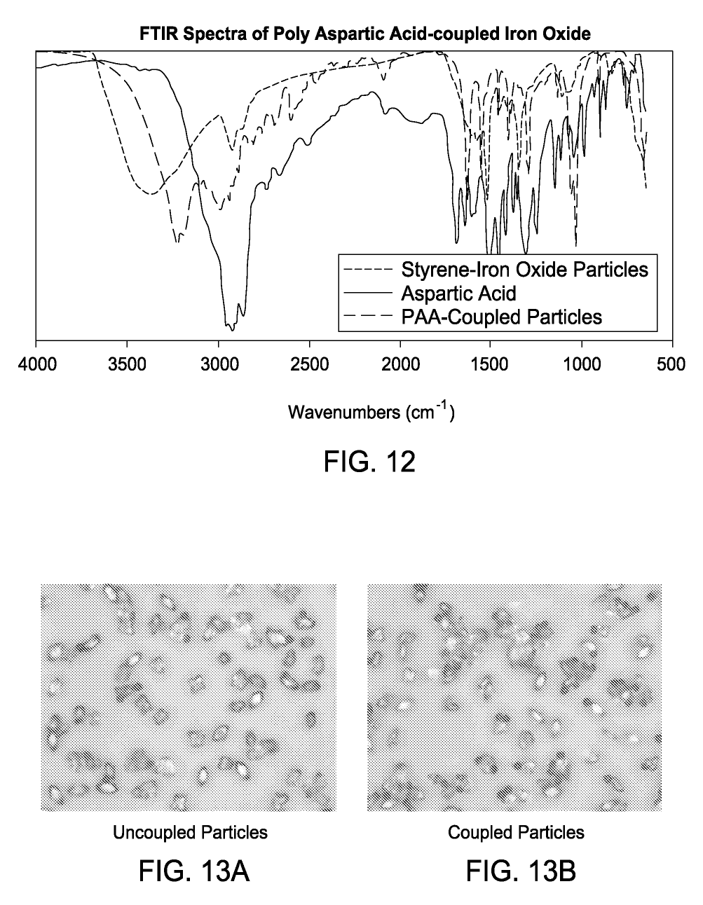

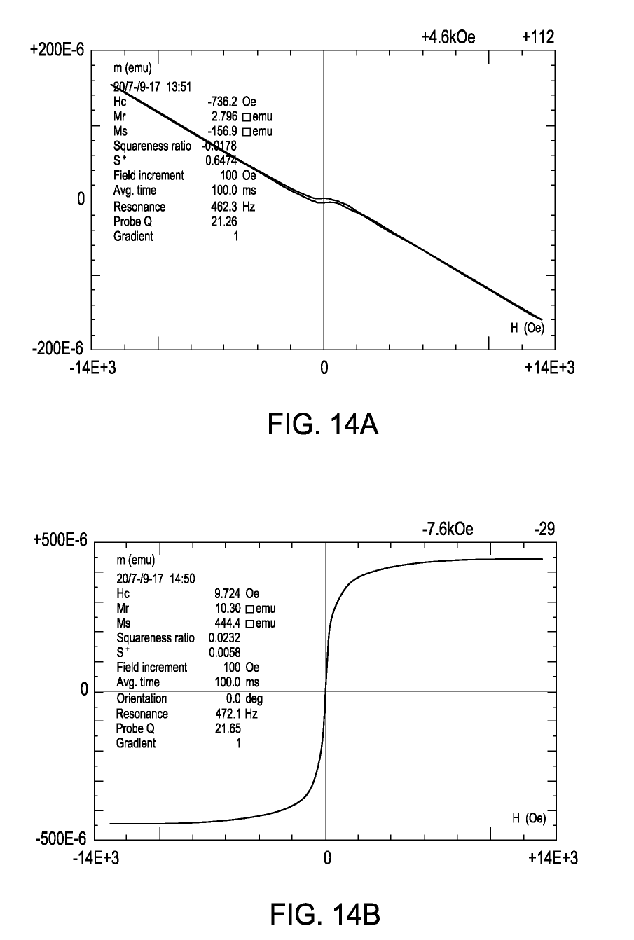

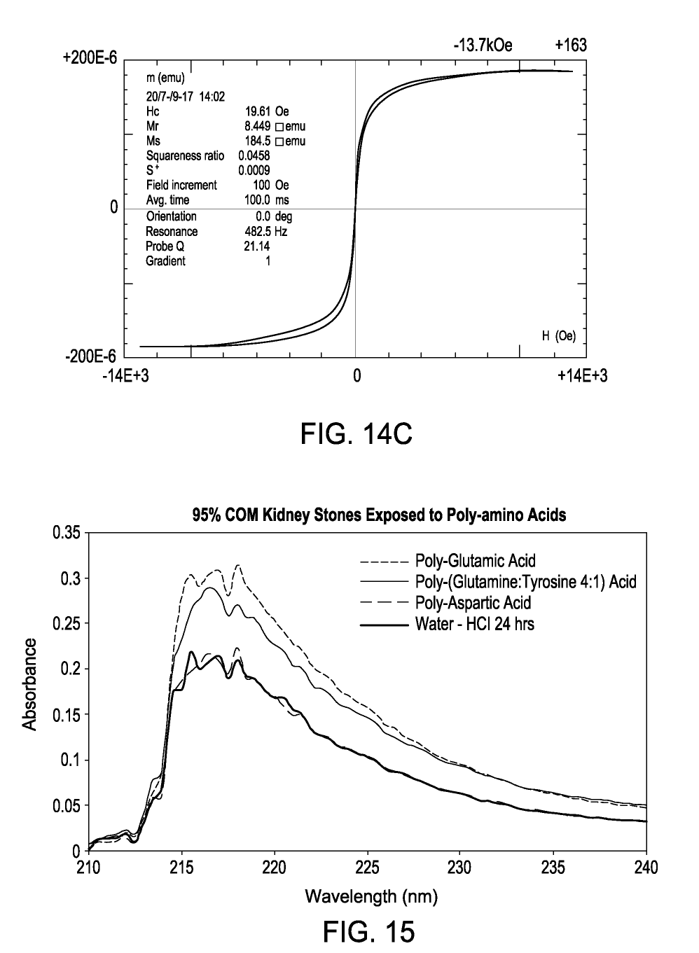

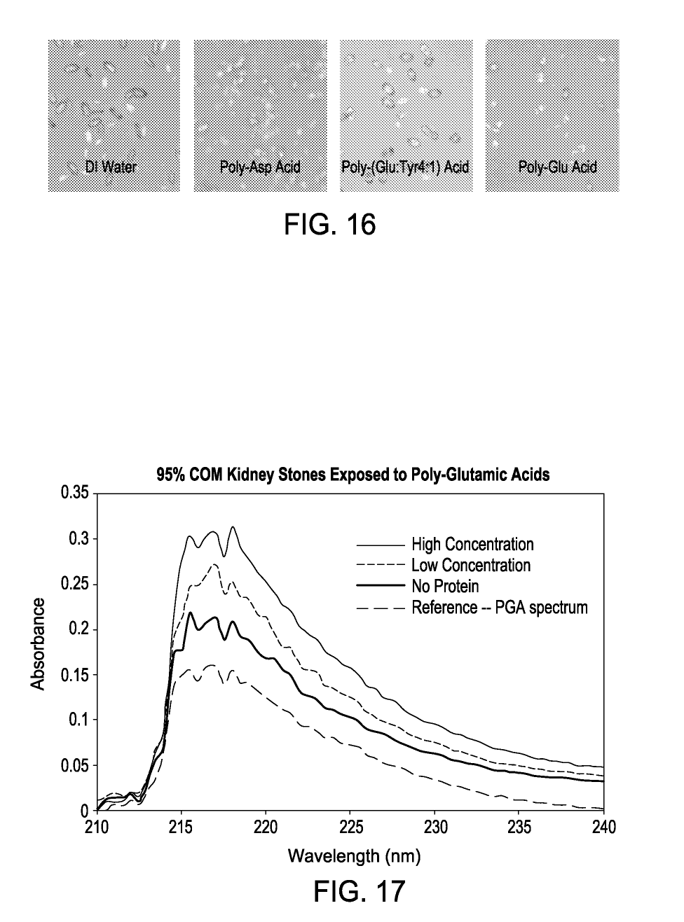

The results from rendering biomaterials magnetic are shown in FIGS. 1-17. Calcium-based biominerals are the primary component of most pathological biomineralization in humans, including atherosclerosis and kidney stones. The ability to deliver targeted therapeutic agents to a biomineralized surface opens a wide variety of treatment, therapy, and imaging options for doctors and surgeons. The functionality of calcium binding proteins was used to develop the compositions and methods of the present invention to selectively attaching micro- and nano-particles to calcium-rich surfaces such as calcium oxalate. The results show selective attachment of iron oxide micro- and nano-particles to calcium-containing biomineral surfaces, although it is anticipated that a wide variety of particles or therapeutic agents could be functionalized using this technique. The resulting functionalized materials are characterized using Scanning and Transmission Electron Microscopy (SEM, TEM), Optical Microscopy, Fourier Transform Infrared Spectroscopy (FTIR) and Alternating Gradient Magnetometry (AGM). Optimization studies were undertaken to increase the selectivity and specificity of the nano and micro particles for such biominerals.

Due to their low solubility, calcium-based biominerals form many of the structural elements of biological organisms. However, these minerals also play a role in several pathologies, including kidney stones and atherosclerosis. While there has been broad, basic research into the role of amino acids and proteins in the creation or prevention of such biomineralization, there has been little or no published research directed towards using proteins to deliver targeted therapies to pathological calcifications in the human body.

It is also possible to use synthetic polypeptides to selectively attach therapeutic particles directly to pathological, calcium-based biominerals. Specifically, it was possible to attach iron oxide particles to Calcium Oxalate Monohydrate, the most common component of kidney stones. It is anticipated that a wide variety of micro- or nanoparticle therapies could be delivered using this technique.

Peptides. Several peptides and protein fragments are known to bind strongly to COM surfaces. For example, a phosphorylated 14-mer derived from the protein osteopontin has shown outstanding ability to attach to COM, inhibiting crystal growth. (attached reference 2008 Phosphorylated OPN bind COM.pdf) In general, a class of peptides known as ‘Aspartic Acid-Rich Peptides’, or AARPs, are also found to strongly bind COM. (attached reference: 2006 Linear AARPs and COM.pdf) Any peptide fragments that bind strongly to COM are a suitable coating for micro- or nanoparticles.

Phage Display. Combinatorial phage display techniques have been used to identify peptide sequences that bind to calcium-based minerals like calcite (source attached: 2004 Gooch dissertation) and to calcium-based biominerals like hydroxyapatite (see patent app referenced in comment). Here, phage display techniques were used to identify peptides that selectively bind to human calcium oxalate kidney stones. Calcium oxalate monohydrate kidney stones extracted from 10 different human patients were combined into a mixture and fragmented into small uniform particles. This stone mixture was used as the target material for a 20-mer phage display library (attached reference: McGuire et al.). Peptide sequences selected from final rounds of phage display show some unique characteristics, including a very high proportion of charged and polar amino acids. More that one third of unique selected peptides contained a methionine at the n-terminus. In particular, two sequences were found to dominate the final round of panning: MGRTVQSGDGTPAQTQPSVN (SEQ ID NO.: 21), designated COM-1, and LRKHADLPGSLSGRVLARPV (SEQ ID NO.: 27), designated COM-2. These peptide sequences, along with peptide sequences that have significant homology with phage-display selected peptides are suitable coating for magnetic particles.

TABLE 3
Phage Display-selected Peptide Sequences.

| | | Sample | | |
|---|---|---|---|---|
| SEQ ID NO.: | Round | Number | Designation | Sequence |
| 1 | 4 | 01 | COM1_R4_01 | YYGPGWYRQFNQGRPGLVTR |
| 2 | 4 | 02 | COM1_R4_02 | AAPHLGRDE------- |
| 3 | 4 | 03 | COM1_R4_03 | MSNASQPAMASDDVLGPG |
| 4 | 4 | 04 | COM1_R4_04 | AIRSAMGNGSPTGAKPWMSW |
| 5 | 4 | 05 | COM1_R4_05 | MGAAFTKPDLKREPSPYSPV |
| 6 | 4 | 06 | COM1_R4_06 | MGMTAKEMREKNGDAPAGAG |
| 7 | 6 | 10 | COM-1 | LRKHADLPGSLSGRVLARPV |
| 8 | 4 | 07 | COM1_R4_07 | SERGRALMPGALAWSTGRVD |
| 9 | 6 | 15 | COM-1 | LRKHADLPGSLSGRVLARPV |
| 10 | 4 | 08 | COM-1 | LRKHADLPGSLSGRVLARPV |
| 11 | 4 | 09 | COM1_R4_09 | MSRGDTGRWDPANPLPGPSE |
| 12 | 4 | 10 | COM-2 | MGRTVQSGDGTPAQTQPSVN |
| 13 | 4 | 11 | COM1_R4_11 | GKVLPRRTGSAPIAYSLARS |
| 14 | 4 | 12 | COM1_R4_12 | GKVVTRQESLLRRGSGTLEV |
| 15 | 4 | 13 | COM1_R4_13 | FSGGRPKPVLQLQREELSSG |
| 16 | 4 | 14 | COM1_R4_14 | MGKPGGVDKTQVQPSSAGHS |
| 17 | 4 | 15 | COM1_R4_15 | TPSEQWVIGPRRTQLGRVAV |
| 18 | 4 | 16 | COM-1 | LRKHADLPGSLSGRVLARPV |
| 19 | 5 | 01 | COM1_R5_01 | MTTAKVDRTRDRHVSDPTKW |
| 20 | 5 | 02 | COM1_R5_02 | MEKVDNYRWSRVWGPQVGKK |
| 21 | 5 | 03 | COM-1 | LRKHADLPGSLSGRVLARPV |
| 22 | 5 | 04 | COM1_R5_04 | MSTGRGLPADPSAKGDVPRN |
| 23 | 5 | 05 | COM1_R5_05 | YVFNLSQAMTRARSAWTGPL |
| 24 | 5 | 06 | COM1_R5_06 | MTKSVEDHAAYDSTESVGSP |
| 25 | 5 | 07 | COM1_R5_07 | GTSRLKIDNPQSTSIGIGAQ |
| 26 | 5 | 08 | COM1_R5_08 | MGTGKTGELKVASKDLGGTP |
| 27 | 5 | 09 | COM-2 | MGRTVQSGDGTPAQTQPSVN |
| | 5 | 10 | COM1_R5_10 | unsequencable |
| 28 | 5 | 11 | COM1_R5_11 | YPAGLGGERMREATGMRGRN |
| 29 | 5 | 12 | COM1_R5_12 | KWTGADLKLSLSSTLPHFRR |
| 30 | 5 | 13 | COM1_R5_13 | LSSNRSAPPSKGEELGMREG |
| 31 | 5 | 14 | COM-2 | MGRTVQSGDGTPAQTQPSVN |
| 32 | 5 | 15 | COM1_R5_15 | MTKSSTPPSRDSEPQATDVG |
| 33 | 5 | 16 | COM-1 | LRKHADLPGSLSGRVLARPV |
| 34 | 6 | 01 | COM1_R6_01 | SAIAVHGGKTARGPGWARLL |
| 35 | 6 | 02 | COM-1 | LRKHADLPGSLSGRVLARPV |
| 36 | 6 | 03 | COM1_R6_03 | TGFRAYLAHQLTSPSWKSSS |
| 37 | 6 | 05 | COM-1 | LRKHADLPGSLSGRVLARPV |
| 38 | 6 | 06 | COM1_R6_06 | MGTSRQGKNETAGVVWASDD |
| 39 | 6 | 07 | COM-1 | LRKHADLPGSLSGRVLARPV |
| | 6 | 08 | COM1_R6_08 | unsequencable |
| 40 | 6 | 09 | COM1_R6_09 | MTKQKMEQTAPDMTPQIDLV |
| 41 | 6 | 12 | COM1_R6_12 | VARFASAREMHKDQAGGVYS |
| 42 | 6 | 13 | COM1_R6_13 | DRPYYQVTRVAPRGSDEVPA |
| 43 | 6 | 14 | COM-1 | LRKHADLPGSLSGRVLARPV |
| 44 | 6 | 16 | COM-1 | LRKHADLPGSLSGRVLARPV |

Other applications. Kidney stones are difficult to image conveniently and accurately using currently available techniques. (attached reference: 2004 Stone imaging) The gold standard for imaging kidney stones is the CT scan, which requires significant time and exposure to radiation. The ability to image kidney stones without using harmful radiation, e.g. with MRI technology, or during a surgical procedure, e.g. with ultrasound or x-ray fluoroscopy would represent a significant advantage over current techniques. Peptides that adhere strongly and selectively to COM can be used to deliver imaging contrast agents, such as MRI contrast agents, ultrasound contrast agents, or X-ray contrast agents to these kidney stones, facilitating diagnosis or treatment. Additionally, the ability of these peptides to inhibit or reverse pathological stone formation offers potential therapeutic applications.

Growth of Synthetic Calcium Oxalate Crystals. Grew a thin layer of thermal SiO₂ on a silicon wafer, evaporated 20 Å Calcium metal onto wafer to seed crystal growth, immersed wafer in a buffered crystal growth solution of sodium oxalate and calcium chloride for 24 hours, and confirmed composition using XRD and GATR-FTIR.

Calcium-Binding Proteins. The classic calcium-binding motif is the EF-hand, which usually contains several carboxylic-rich residues (Glu, Asp) along with Glycine, which provides flexibility. Other proteins that interact with calcium-based biominerals, such as osteopontin and prothrombin fragment, are generally classified as carboxylic acid-rich proteins. Small carboxyl-containing molecules such as citrate are known to have an inhibitory effect on the growth of Calcium Oxalate crystals in healthy urine.

COM and Carboxyl-functionalized Nanoparticles. Evaporated 100 Å SiO₂ followed by 100 Å Calcium metal onto copper TEM grids, immersed grids in COM crystal growth solution for 24 hours, exposed grids to solutions of commercially available iron oxide nanoparticles with carboxyl-functionalized surfaces for 24 hours and rinsed with saline.

Polypeptide Functionalized Microparticles. Attached Poly-aspartic acid (PAA) to commercially available iron oxide microparticles using a standard carbodiimide coupling protocol. Confirmed attachment using FTIR. Exposed synthetic COM crystals to iron oxide particles in solution for 30 minutes, rinsed wafers with DI water and then dried with nitrogen prior to examination.

COM Selectivity Studies: Poly-Amino Acids. Selective Crystal Etching. Exposed 95% COM Kidney Stones to solutions containing equal concentrations of poly-amino acids, rinsed stones, then etched in 3% HCl for 24 hours and then measured UV-Vis Absorbance of etched solution. Etch rate vs. concentration was determined by exposing 95% COM Kidney Stones to solutions containing varying concentrations of poly-glutamic acid, then rinsed the Stones, then etched in 3% HCl for 24 hours and measured UV-Vis Absorbance of etched solution.

### EXAMPLE 3

### Movement of Kidney Stone Fragments by Attachment of Nanocomposite Superparamagnetic Particles

Large renal or ureteral calculi that are unable to pass spontaneously require endoscopic fragmentation or shock wave lithotripsy (SWL). While endoscopy has the advantage of manual fragment retrieval to improve stone free rates, access to these fragments may be hindered by collecting system anatomy, technical limitations of the endoscope, or poor visibility. Furthermore, retrieving the entire burden of stone fragments, while desirable, is time-consuming. The ability to attract and move these fragments en masse into an easily accessible area of the collecting system could facilitate endoscopic stone surgery as well as diminish costs by reducing time and obviating the need for secondary procedures. Novel nanocomposite paramagnetic particles were developed that selectively adhere to the calcium oxalate crystalline structure of a stone such that introduction of a magnetized instrument and permit attraction of dispersed stone fragments into a favorable location for removal.

Functionalization: Human calcium-based kidney stones were fragmented to 1-5 millimeters and incubated with 1 micron iron oxide particles with a poly-aspartic acid protein coat for varying time durations. The solutions were then exposed to magnets of differing sizes Urothelial interaction: a solution of 1 micron carboxy-terminated magnetic beads were injected directly into mouse bladders. After incubation periods of 5 (n=4) and 10 minutes (n=4), bladders were harvested and fixed in formalin (saline used for negative control). Freshly harvested urothelial strips with the Fe₂O₃ solution applied directly to apical surface served as the positive control (H&E and Prussian blue stains).

Functionalization conditions. The default environment for functionalizing stone fragments in vivo is incubating the fragments and stone particles together in a isotonic saline solution (0.9% NaCl in pure water). However, the ionic strength of the saline solution has an impact on the required incubation time. We have also found that storage buffers used to store the functionalized particles play a significant role in reducing or increasing incubation time. The combination of particle coating, particle concentration, incubation fluid, and buffer system can be optimized to reduce incubation time. For example, we find that 1-micron particles coated with poly-aspartic acid can be incubated in phosphate-buffered saline (PBS) with fragmented kidney stones for 5 minutes, and after this brief time, stone fragments can be retrieved from a simulated bladder with a small magnetized surgical instrument.

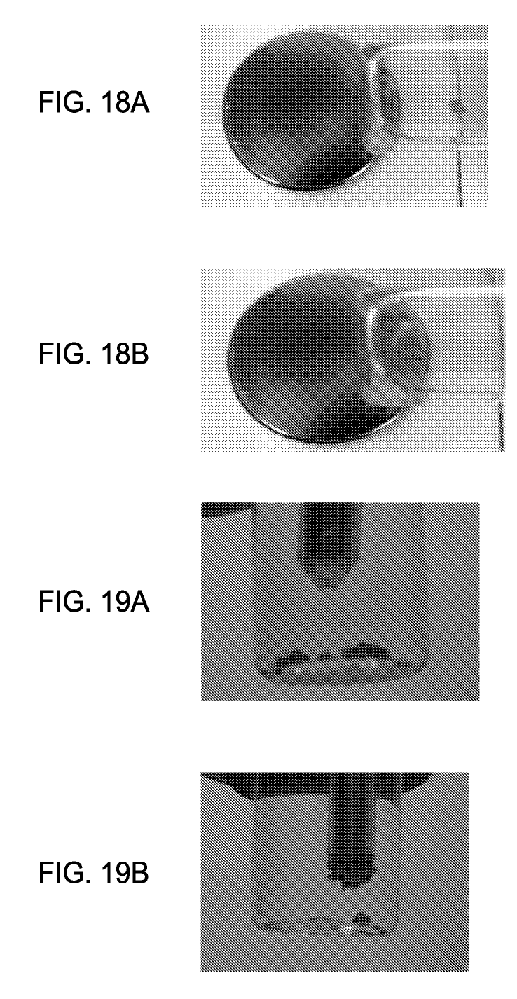

FIGS. 18A and 18B show that a magnet approaching 3-4 mm stone fragments (18A); magnet attracts functionalized stone fragments from 1 cm (18B). FIGS. 19A and 19B shows that a magnet approaching 1-2 mm stone fragments (19A); magnet with attached functionalized stone fragments (19B).

These figures demonstrate that magnetic particles stably adhered to the stone fragments. A small handheld magnet at a distance of 1 cm moved 3-4 mm stone fragments (FIGS. 18A and 18B). A smaller (3 mm diameter×1 cm) magnet moved 1-2 mm stone fragments from shorter distances away (FIGS. 19A and 19B). Small stone fragments were able to be magnetically attracted after incubation times as brief as 5 minutes. Magnetic particles were not seen on H&E or Prussian blue stains in the negative control group or in the 5 and 10 minute magnetic particle incubation cohorts. The positive controls revealed magnetic particles.

Therefore, iron oxide-coupled kidney stone fragments can be magnetically manipulated using small permanent magnets. Conversely, magnetic particles can be bound selectively to stone fragments, in either case, the particle (whether ferrous or magnetic) should not to urothelial surfaces (or with the least amount of binding possible). Magnetic particles with larger magnetic moments and improved surface coatings can also be used to facilitate use of smaller magnets, reduce incubation times, and maintain selectivity for kidney stones over urothelium.

### EXAMPLE 4

### Magnet Field Applications

The examples above show the development of paramagnetic micro- and nanoparticles that can selectively attach to calcium stones. Once the stone or stone fragments are magnetized by the instillation of these microparticles, the surgeon has the further option of manipulating the stone fragments by the application of an external magnetic field. The magnetic field can be generated by one or more permanent or electromagnets. The one or more magnetic fields can be generated by magnets that are individually hand-held or supported using a custom-made arm (manual or robotic). The optimal design of the magnets may include engineering solutions such as “magnetic flux focusing.” The size specifications will depend on the needed magnetic force and will depend on patient size and distance from skin to stones. The size and power of the magnetic field applied to the target tissue using magnetic tools (or a combination with internal magnetic tools) will depend on the size of the target fragments, extent of coating, and friction encountered at the target size.

Magnetic tools are applied, directed and focused in such a manner that stones or stone fragments can be manipulated and moved by the surgeon to different locations within the urinary collecting system (calyx, pelvis, ureter or bladder). It may be possible for the surgeon to move the stone from the kidney or ureter through the collecting system and into the bladder/urethra. It is also envisioned that during a ureteroscopy or percutaneous nephrolithotomy (through the back), the surgeon will be able to magnetically move the stone fragments into an position for favorable stone extraction. For example, in some cases the surgeon may not be able to grasp the stone with a basket or grasper due to a poor angle, but with magnetization, the surgeon will be able to move the stone to allow easy grasping and removal.

Interaction between ferromagnetic particles and urothelium. Following Institutional Animal Care and Use Committee (IACUC) approval, we assessed the affinity of Fe2O3 particles for murine and porcine urothelium. The mice were anesthetized and the bladders were dissected and exposed. A commercially available solution of 1 micron long-arm carboxy-terminated magnetic beads was injected directly into mouse bladders. White gauze was placed at the urethral meatus to ensure that the solution remained in the bladder. After incubation periods of 5 minutes (n=4) and 10 minutes (n=4), the bladders were harvested and fixed in formalin. Saline (n=4) injection with incubation time of 10 minutes served as the negative control. All specimens were processed using hematoxylin and eosin (H&E) and Prussian blue stains and reviewed by a uropathologist.

Three freshly harvested porcine urothelial strips with 5 ml of the Fe2O3 solution applied directly to the apical surface for a period of 10 minutes served as the positive control. The first strip was analyzed directly, the second was washed once with 15 cc of normal saline, and the third strip was washed twice with 15 cc of normal saline before formalin fixation. Specimens were first viewed under a tissue microscope immediately after incubation and washing and then fixed in formalin for H&E and Prussian blue review.

It was found that the particles did not bind significantly to any of the tissues. For mouse urothelium the magnetic particles were not seen on H&E or Prussian blue stains in the negative control group or in the 5 and 10 minute magnetic particle incubation cohorts.

For Porcine urothelium it was found under the tissue microscope that the magnetic particles were seen with decreasing density after serial washings and no iron oxide particles were seen on H&E or Prussian blue stains after the washes were completed.

The compositions and methods of the present invention were used to successfully demonstrated that micro- and nano-particles can be attached to calcium-based biominerals. Further studies can focus on improving the protein coating on the particles to enhance selectivity for specific biominerals, utilizing the quantitative methods outlined here.

### EXAMPLE 5

### Attachment of Fluorescent Magnetic Particles to Kidney Stones for Detection and Retrieval and Tools to Find and Retrieve Particles

This Example demonstrates that fluorescent materials can be attached to small kidney stone fragments following fragmentation to help surgeons visualize and remove all pieces using specialized instruments, thus reducing the chance of recurrence.

While this is a great advancement in the collection of kidney stone fragments, there is still a need to be able to detect stone fragments so the surgeon can be sure that all of them have been removed. The invention presented in this disclosure is the idea that either the magnetic particles or the proteins used to attach the magnetic particles to the kidney stones could be made fluorescent. When the particles are illuminated with a specific wavelength of light, they will fluoresce at a different, unique wavelength that is not present in the background illumination, making the stone fragments visible to the surgeon. Fluorescent materials may be selected with absorption wavelengths that are not strongly absorbed by the saline solution and blood that is present in the kidney during the surgical procedure, such as in the ultraviolet region. In certain embodiment, the emitted light is a unique color that is easily distinguished from the background-reflected light in the kidney and is at a wavelength that is not strongly absorbed by the solution environment in the kidney during the procedure. A light source is selected of the proper wavelength and intense enough to fluoresce the functionalized stone fragments so the surgeon can see them through the endoscope, e.g., a light emitting diode of the proper wavelength may be positioned to transmit the light through the endoscope or the LEDs can be positioned at the tip or delivered through the endoscope. An organic light emitting diode (OLED) would have the advantage that an annular light source that is very thin could be added to the end of the endoscope, which could be turned on only when looking for fragments. The OLED light source could be multiple colors so that it could be used for general illumination during the procedure, as well as for exciting the particles.

By exciting the target particles optically, the amount of x-ray radiation the patient receives is decreased because the need to use fluoroscopy to look for small stone fragments during the surgical procedure will be reduced or eliminated. The ability to tune both the photoluminescent absorption and emission energy allow the user to use excitation energies that are not strongly absorbed by the bulk of the materials present during the procedure, greatly increasing the sensitivity of the technique. Also, using an annular OLED tunable light source should reduce the amount of power required to illuminate the working environment during the procedure, decreasing the chance of bringing a hot surface in contact with the tissue. The fluorescent tag may be included as part of the protein that is used to attach the magnetic particles to the stone fragments. Generally, enough fluorescent material is attached to the stone fragments to provide adequate sensitivity, photoluminescent material, either organic or inorganic, could also be added to the polystyrene spheres in larger quantities. There is a wide variety of inorganic photoluminescent phosphors that could be used by simply incorporating the phosphor material in the polystyrene spheres with the magnetic material.

In operation, polystyrene spheres with iron oxide particles can be made depending on the fluorescent material used. Examples of materials for use with the present invention include photoluminescent phosphors. The specific phosphor can be determined by the required absorption and emission energies.

Suitable labels or dyes or fluorophores include, without limitation, any atomic element amenable to attachment to a specific site in a polymerizing agent or dNTP, especially fluorescent dyes such as d-Rhodamine acceptor dyes including dichloro[R110], dichloro[R6G], dichloro[TAMRA], dichloro[ROX] or the like, fluorescein donor dye including fluorescein, 6-FAM, or the like; Acridine including Acridine orange, Acridine yellow, Proflavin, or the like; Aromatic Hydrocarbon including 2-Methylbenzoxazole, Ethyl p-dimethylaminobenzoate, Phenol, Pyrrole, benzene, toluene, or the like; Arylmethine Dyes including Auramine O, Crystal violet, Crystal violet, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dye including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, Thiacarbocyanine (C3) dye, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), 4-Dimethylamino-4′-nitrostilbene, Merocyanine 540, or the like; Miscellaneous Dye including 4′,6-Diamidino-2-phenylindole (DAPI), 4′,6-Diamidino-2-phenylindole (DAPI), 7-Benzylamino-4-nitrobenz-2-oxa-1,3-diazole, Dansyl glycine, Dansyl glycine, Hoechst 33258, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, Nile Red, Nile blue, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridyl)ruthenium(II), Tetrapyrrole, Bilirubin, Chlorophyll a, Chlorophyll a, Chlorophyll b, Diprotonated-tetraphenylporphyrin, Hematin, Magnesium octaethylporphyrin, Magnesium octaethylporphyrin (MgOEP), Magnesium phthalocyanine (MgPc), Magnesium phthalocyanine (MgPc), Magnesium tetramesitylporphyrin (MgTMP), Magnesium tetraphenylporphyrin (MgTPP), Octaethylporphyrin, Phthalocyanine (Pc), Porphin, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, Fluorescein, Rhodamine 123, Rhodamine 6G, Rhodamine B, Rose bengal, Sulforhodamine 101, or the like; or mixtures or combination thereof or synthetic derivatives thereof or FRET fluorophore-quencher pairs including DLO-FB1 (5′-FAM/3′-BHQ-1) DLO-TEB1 (5′-TET/3′-BHQ-1), DLO-JB 1 (5′-JOE/3′-BHQ-1), DLO-HB 1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DL0-C5B3 (5′-Cy5/3′-BHQ-3), DL0-C55B3 (5′-Cy5.5/3′-BHQ-3), MBO-FB1 (5′-FAM/3′-BHQ-1), MBO-TEB1 (5′-TET/3′-BHQ-1), MBO-JB1 (5′-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif. or any other fluorescent donor or acceptor. Suitable labels also include quantum dots, or other persistent nano-structured fluorophores.

Other examples of fluorescent dye include Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide(Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide (PI) and derivatives thereof and Cy-dyes derivatives.

Examples of proteins that are fluorescent include blue fluorescent protein (BFP), green fluorescent protein (GFP), photo activatable-GFP(PA-GFP), yellow shifted green fluorescent protein (Yellow GFP), yellow fluorescent protein (YFP), enhanced yellow fluorescent protein (EYFP), cyan fluorescent protein (CFP), enhanced cyan fluorescent protein (ECFP), monomeric red fluorescent protein (mRFP1), kindling fluorescent protein (KFP1), aequorin, autofluorescent proteins (AFPs), JRed, TurboGFP, PhiYFP and PhiYFP-m, tHc-Red (HcRed-Tandem), PS-CFP2 and KFP-Red.

During a surgical procedure using the present invention it was possible to see stone fragments under the current scope illumination, e.g., the orange shade of the ferrous particle was enough to visualize stone dust. For larger stone fragments, e.g., larger than 0.25 mm, the surgeon can attract and remove the stones manually. The present invention also allows the surgeon to visualize stone ‘dust’ in the surgical field. For example, after the surgeon finishes the removal of larger stone fragments, they turn off the visible white light of the scope and turn on a narrow spectrum light source (like an OLED) to ‘vacuum up’ the remaining stone dust under fluorescent or polarized light visualization using a magnetic wand or other magnetic instrument. A dye, visible or fluorescent, allows the surgeons to ensure that no magnetic particles are being left inside the kidneys and maximize the removal of possible stone fragments or dust that could serve as re-nucleation sites.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. M. Menon, B. Parulkar, G. Drach: Urinary lithiasis: Etiology, diagnosis and medical management. In Walsh P, Retik A, Darracott E, et al (eds): Campbell's Urology, ed 7. Philadelphia, W B Saunders, 1998, pp 2659-2733
2. M. S. Pearle, E. A. Calhoun and G. C. Curhan, “The Urological Diseases in America Project Urolithiasis”, J. Urol. 173, 848 (2005).
3. A. Jakubowicz, H. Jia, R. M. Wallace and B. Gnade, “Adsorption Kinetics of p-Nitrobenzenethiol Self-Assembled Monolayers on a Gold Surface”, Langmuir 21, 950-955 (2005).
4. F. C. Delvecchio and G. Preminger, “Management of residual stones”, Uro. l Clin. North Am. 27, 347 (2000).
5. T. Nakamoto, K. Sagami, A. Yamasaki A, et al, “Long-term results of endourologic treatment of urinary calculi: Investigation of risk factors for recurrence or regrowth”, J. Endourol. 7, 297 (1993).
6. D. J. Galvin and M. S. Pearle, “The contemporary management of renal and ureteric calculi” BJU Int 98, 1283 (2006).
7. M. D. Ward, “Snapshots of Crystal Growth”, Science 308, 1566 (2005).
8. X. Sheng, M. D. Ward, J. A. Wesson, “Crystal Surface Adhesion Explains the Pathological Activity of Calcium Oxalate Hydrates in Kidney Stone Formation”, J. Am, Soc. Nephrol. 16, 1904 (2005).
9. X. Sheng, T. Jung, J. A. Wesson and M. D. Ward, “Adhesion at calcium oxalate crystal surfaces and the effect of urinary constituents”, Proc. NAS 102, 267 (2005).
10. Q. A. Pankhurst, J. Connolly, S. K. Jones and J. Dobson, “Applications of magnetic nanoparticles in biomedicine”, J. Phys. D: Appl. Phys. 36, R167 (2003).
11. P. Gould, “Nanomagnetism shows in vivo potential”, NanoToday 1, 35 (2006).
12. P. Tartaj, M. del Puerto Morales, S. Veintemillas-Verdaguer, T. Gonzalez-Carreno and C. J. Serna, “The preparation of magnetic nanoparticles for application in biomedicine”, J. Phys. D: Appl. Phys. 36, R182 (2003).
13. C. C. Berry and A. S. G. Curtis, “Functionalisation of magnetic nanoparticles for applications in biomedicine”, J. Phys. D: Appl. Phys. 36, R198 (2003).
14. A. K. Gupta and M. Gupta, “Synthesis and surface engineering of iron oxide nanoparticles for biomedical applications”, Biomaterials 26, 3995 (2005).
15. D. I. Kreller, G. Gibson, G. W. vanLoon, J. H. Horton, “Chemical Force Microscopy Investigation of Phosphate Absorption on the surfaces of Iron (III) oxyhydroxide particles”, J. Colloid Interface Sci. 254, 205 (2002).
16. I. Gouzman, M. Dubey, M. D. Carolus, J. Schwartz and S. Bernasek, “Monolayer vs. multilayer self-assembled alkylphosphonate films: X-ray Photoelectron Spectroscopy studies”, Surf. Sci 600, 773 (2006).
17. Y. G. Aronoff, B. Chen, L. Gang, C. Seto, J. Schwartz and S. L. Bernasek, “Stabilization of self-assembled monolayers of carboxylic acids on native oxides of metals”, J. Am. Chem. Soc. 119, 259 (1997).

What is claimed is:

1. A method of magnetizing a biological particle comprising:
contacting a biological particle with a ferrous or magnetic particle that is able to specifically bind the biological particle; and
reacting, adsorbing, or adhering the biological particle with the ferrous or magnetic particle under physiological conditions, wherein the ferrous or magnetic particle causes the biological particle to become attractable magnetically.

2. The method of claim 1, wherein the biological particle comprises a kidney stone or fragment thereof.

3. The method of claim 1, wherein the ferrous or magnetic particle further comprises an agent that specifically binds the surface of the biological target with high affinity such as antibodies, aptamers, peptides, polypeptides, proteins, protein fragments, amino acids, polyamino acids, phosphonic acids, carboxylic acids, long chain phosphonic and long chain carboxylic acids.

4. The method of claim 1, wherein the biological particle comprises at least one of Apatite, Calcium Oxalate Dihydrate, Calcium Oxalate Monohydrate, Struvite, Cysteine, Brushite and Uric Acid.

5. The method of claim 1, wherein the ferrous particle comprises Fe, FeC, Fe₂O₃ or Fe₃O₄.

6. The method of claim 1, wherein the biological particle comprises a kidney stone or fragment thereof coated with a ferrous particle and the particle is moved within a lumen using a magnet.

7. The method of claim 1, wherein the ferrous or magnetic particle is suspended in saline.

8. The method of claim 1, wherein the biological particles and the ferrous or magnetic particles are located in a anatomical lumen and further comprising the step of removing the biological particle with a device that attracts the ferrous or magnetic particle.

9. The method of claim 1, wherein the ferrous or magnetic particles are between 10 nm to 1 mm.

10. The method of claim 1, wherein the ferrous or magnetic particle further comprises an agent that specifically binds the surface of the biological target selected from proteins that interact with calcium-based biominerals, such as carboxylic acid-rich proteins, osteopontin and prothrombin fragment 1; small carboxyl-containing molecules such as citrate are known to have an inhibitory effect on the growth of Calcium Oxalate crystals in healthy urine; and calcium-binding motif proteins that includes the EF-hand, which have carboxylic-rich residues (Glu, Asp) and Glycine for flexibility.

11. A medical device, comprising:
an expansible member having a proximal end, a delivery state, and a deployed state; wherein the deployed state comprises an at least partially magnetic portion for deployment within an anatomical lumen for the capture of a magnetic target material, and wherein the deployed state is configured to retrieve the material from within the anatomical lumen.

12. The medical device of claim 11, further comprising an elongated flexible tube including a distal end and a proximal end, the tube defining a channel extending from the proximal end of the tube to an aperture at the distal end and wherein the deployed portion of the member is housed within the channel prior to deployment within the anatomical lumen.

13. The medical device of claim 11, wherein the delivery state is a compressed state.

14. The medical device of claim 11, wherein the device extends proximally out of the channel and is configured to control axial movement of the expansible member relative to the tube.

15. The medical device of claim 11, wherein the deployed state is further defined as expansible, wherein the expansible state that has a proximal end and a distal end, and markers are positioned proximate at least one of the distal and proximal ends of the expansible member.

16. The medical device of claim 11, wherein the expansible member has a tapered proximal end to facilitate releasable engagement with a distal end of the instrument.

17. The medical device of claim 11, wherein the expansible member includes a protrusion at the proximal end to facilitate engagement between the instrument and the expansible member.

18. The medical device of claim 11, wherein the expansible member comprises a material that exhibits an expansion/compression size ratio of approximately 10:1.

19. The medical device of claim 11, wherein the expansible member comprises a biocompatible polymer, plastic, nylon, polyester, or metal.

20. The medical device of claim 11, wherein the expansible member comprises a cavity.

21. The medical device of claim 11, wherein the expansible member defines holes formed therein for passing irrigation therethrough in the deployed state.

22. The medical device of claim 11, wherein the device further comprises grasping forceps, a collapsible basket, a hook, a net, a lasso, or a sponge.

23. The medical device of claim 11, wherein the expansible member expands to fill a cross-sectional area of an anatomical lumen in the expanded, deployed state.

24. A method for immobilizing a magnetic biological material in a body comprising:
inserting a magnetic expansible member into an anatomical lumen of the body, the expansible member having a delivery state, an expanded state, and a proximal end detachably engaged with a distal end of an instrument;
positioning the instrument to deploy the expansible member such that the expansible member transforms from the delivery state to the expanded state at a treatment site within the anatomical lumen; and
capturing biological particles within the lumen that have been modified in situ to be attracted magnetically.

25. The method of claim 24, wherein inserting an expansible member includes providing an elongated flexible tube including a distal end and a proximal end, the tube defining a channel extending from the proximal end of the tube to an aperture at the distal end, and wherein the expansible member is housed within the channel prior to deployment at a treatment site.

26. The method of claim 24, wherein positioning the instrument to deploy the expansible member includes moving the instrument relative to the tube to control axial movement of the expansible member beyond the channel.

27. The method of claim 24, wherein the expansible member comprises a material that expands to the expanded state when unrestrained.

28. The method of claim 24, wherein the expansible member is deployed distally beyond the material to be immobilized such that the expansible member at least partially occludes the anatomical lumen.

29. The method of claim 24, further comprising performing a lithotripsy procedure on the biomaterial.

30. The method of claim 24, further comprising irrigating the anatomical lumen with a composition comprising a ferrous or magnetic particle that is able to specifically bind the biological material.

31. The method of claim 24, further comprising retrieving the immobilized biological material by proximally pulling the expansible member through the anatomical lumen.

32. The method of claim 24, wherein the anatomical lumen includes an interior surface and the expansible member expands to contact the interior surface of the anatomical lumen.

33. The method of claim 24, wherein the expansible member has a proximal end and a distal end, and markers are positioned proximate the distal and proximal ends of the expansible member.

34. The method of claim 33, wherein positioning further includes visualizing the position of the markers through a medical imaging device.

35. The method of claim 24, further comprising retrieving the immobilized material by engaging fragmented immobilized material with the device.

36. A method for stabilizing a target biomaterial in a patient's body comprising:
contacting a first material comprising a magnetic portion with a target biomaterial to cause the biomaterial to become magnetic in a body lumen under physiologic conditions;
inserting a medical device comprising a magnetically attracting end, wherein the magnetized biomaterial is attracted to the medical device; and
removing the biomaterial from the patient's body.

37. The method of claim 36, wherein the target biomaterial is broken into at least two fragments by technique selected from the group consisting of extra-corporeal shock wave lithotripsy, intra-corporeal shock wave lithotripsy, or Holmium laser fragmentation.

38. A kit comprising:
one or more containers comprising a ferrous or magnetic particle that is able to specifically bind to a biological particle, and
one or more devices comprising an end that is magnetic and able to attract the ferrous or magnetic particle in a body lumen.

39. A method of magnetizing and moving a biological particle comprising:
contacting a biological particle with a ferrous particle that is able to specifically bind the biological particle;
reacting, adsorbing, or adhering the biological particle with the ferrous particle under physiological conditions, wherein the ferrous or magnetic particle causes the biological particle to become attractable magnetically; and
directing a magnet to the location of the ferrous particle to move the particle using the magnetic field generated by the magnet.

40. The method of claim 39, wherein the biological particle comprises a kidney stone or fragment thereof.

41. The method of claim 39, wherein the ferrous particle further comprises an agent that specifically binds the surface of the biological target with high affinity such as antibodies, aptamers, peptides, polypeptides, proteins, protein fragments, amino acids, polyamino acids, phosphonic acids, carboxylic acids, long chain phosphonic and long chain carboxylic acids.

42. The method of claim 39, wherein the biological particle comprises at least one of Apatite, Calcium Oxalate Dihydrate, Calcium Oxalate Monohydrate, Struvite, Cysteine, Brushite and Uric Acid.

43. The method of claim 39, wherein the ferrous particle comprises Fe, FeC, Fe₂O₃ or Fe₃O₄.

44. The method of claim 39, wherein the biological particle comprises a kidney stone or fragment thereof.

45. The method of claim 39, wherein the biological particles and the ferrous or magnetic particles are located in a anatomical lumen and further comprising the step of removing the biological particle with a device that attracts the ferrous or magnetic particle.

46. A method of identifying a biological particle comprising:
contacting a biological particle with a ferrous particle that is able to specifically bind the biological particle and a dye;
reacting, adsorbing, or adhering the biological particle with the ferrous particle under physiological conditions, wherein the ferrous or magnetic particle causes the biological particle to become attractable magnetically;
directing an magnet to the location of the ferrous particle to move the particle using the magnetic field generated by the magnet; and
pointing a light that excites the dye to provide visualization of the particles.

47. The method of claim 46, wherein the dye comprises a fluorescent dye.

48. The method of claim 46, wherein the dye comprises a visible dye.

49. The method of claim 46, wherein the dye comprises a fluorescent dye selected from the group of Acridine homodimer and derivatives thereof, Acridine Orange and derivatives thereof, 7-aminoactinomycin D and derivatives thereof, Actinomycin D and derivatives thereof, 9-amino-6-chloro-2-methoxyacridine (ACMA) and derivatives thereof, DAPI and derivatives thereof, Dihydroethidium and derivatives thereof, Ethidium bromide and derivatives thereof, EthD-1 and derivatives thereof, EthD-2 and derivatives thereof, Ethidium monoazide and derivatives thereof, Hexidium iodide and derivatives thereof, bisbenzimide (Hoechst 33258) and derivatives thereof, Hoechst 33342 and derivatives thereof, Hoechst 34580 and derivatives thereof, hydroxystilbamidine and derivatives thereof, LDS 751 and derivatives thereof, Propidium Iodide (PI) and derivatives thereof and Cy-dyes derivatives.

50. The method of claim 46, wherein the dye is selected from a group consisting of blue fluorescent protein (BFP), green fluorescent protein (GFP), photo activatable-GFP(PA-GFP), yellow shifted green fluorescent protein (Yellow GFP), yellow fluorescent protein (YFP), enhanced yellow fluorescent protein (EYFP), cyan fluorescent protein (CFP), enhanced cyan fluorescent protein (ECFP), monomeric red fluorescent protein (mRFP1), kindling fluorescent protein (KFP1), aequorin, autofluorescent proteins (AFPs), JRed, TurboGFP, PhiYFP and PhiYFP-m, tHc-Red (HcRed-Tandem), PS-CFP2 and KFP-Red.

51. The method of claim 46, further comprising the step of removing dyed kidney stone dust.

52. The method of claim 46, wherein the surgical field is illuminated with polarized light to maximize the visualization of dyed kidney stone dust.
